(19) United States
(12) Patent Application Publication
(10) Pub. No.: US 2011/0313001 A1
(43) Pub. Date: Dec. 22, 2011

(54) TRIAZOLE DERIVATIVES FOR TREATMENT OF ALZHEIMER'S DISEASE

(76) Inventors: Christian Fischer, Natick, MA (US); Adam J. Schell, Newton, MA (US); Benito Munoz, Cambridge, MA (US)

(21) Appl. No.: 13/133,833

(22) PCT Filed: Dec. 7, 2009
(86) PCT No.: PCT/US2009/066906

§ 371 Date: Aug. 23, 2011

Related U.S. Application Data
(60) Provisional application No. 61/122,960, filed on Dec. 16, 2008.

(51) Int. Cl. A61K 31/4439; A61P 25/28; C07D 403/10; A61K 31/4192; C07D 401/10; C07D 413/14

(52) U.S. Cl. 514/338; 546/268.4; 546/271.7; 548/255; 514/340; 514/406

(57) ABSTRACT

According to the invention there is provided a compound of formula (I): (I) or a pharmaceutically acceptable salt or hydrate thereof; wherein the variables are as defined herein. The compounds selectively attenuate the production of Aβ42 and hence are useful in treatment of Alzheimer's disease and related conditions.

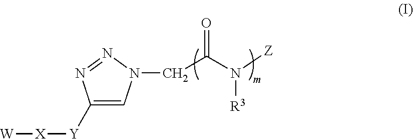

This invention relates to compounds for use in therapeutic treatment of the human body. In particular, it provides triazole derivatives useful for treating diseases associated with the deposition of β-amyloid peptide in the brain, such as Alzheimer's disease, or of preventing or delaying the onset of dementia associated with such diseases.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. Variability in the site of the proteolysis mediated by γ-secretase results in Aβ of varying chain length, e.g. Aβ(1-38), Aβ(1-40) and Aβ(1-42). N-terminal truncations such as Aβ (4-42) are also found in the brain, possibly as a result of variability in the site of proteolysis mediated by β-secretase. For the sake of convenience, expressions such as “Aβ(1-40)” and “Aβ(1-42)” as used herein are inclusive of such N-terminal truncated variants. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Other dementing conditions associated with deposition of Aβ in the brain include cerebral amyloid angiopathy, hereditary cerebral haemorrhage with amyloidosis, Dutch-type (HCHWA-D), multi-infarct dementia, dementia pugilistica and Down syndrome.

Various interventions in the plaque-forming process have been proposed as therapeutic treatments for AD (see, for example, Hardy and Selkoe, Science, 297 (2002), 353-6). One such method of treatment that has been proposed is that of blocking or attenuating the production of Aβ for example by inhibition of β- or γ-secretase. It has also been reported that inhibition of glycogen synthase kinase-3 (GSK-3), in particular inhibition of GSK-3α, can block the production of Aβ (see Phiel et al, Nature, 423 (2003), 435-9). Other proposed methods of treatment include administering a compound which blocks the aggregation of Aβ, and administering an antibody which selectively binds to Aβ.

However, recent reports (Pearson and Peers, J. Physiol., 575.1 (2006), 5-10) suggest that Aβ may exert important physiological effects independent of its role in AD, implying that blocking its production may lead to undesirable side effects. Furthermore, γ-secretase is known to act on several different substrates apart from APP (e.g. notch), and so inhibition thereof may also lead to unwanted side effects. There is therefore an interest in methods of treating AD that do not suppress completely the production of Aβ, and do not inhibit the action of γ-secretase.

One such proposed treatment involves modulation of the action of γ-secretase so as to selectively attenuate the production of Aβ(1-42). This results in preferential secretion of the shorter chain isoforms of Aβ, which are believed to have a reduced propensity for self-aggregation and plaque formation, and hence are more easily cleared from the brain, and/or are less neurotoxic. Compounds showing this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70). Compounds which modulate the activity of PPARα and/or PPARδ are also reported to have the effect of lowering Aβ(1-42) (WO 02/100836). NSAID derivatives capable of releasing nitric oxide have been reported to show improved anti-neuroinflammatory effects and/or to reduce intracerebral Aβ deposition in animal models (WO 02/092072; Jantzen et al, J. Neuroscience, 22 (2002), 226-54). US 2002/0015941 teaches that agents which potentiate capacitative calcium entry activity can lower Aβ(1-42).

Further classes of compounds capable of selectively attenuating Aβ(1-42) production are disclosed in WO 2005/054193, WO 2005/013985, WO 2006/008558, WO 2005/108362, WO 2006/043064. WO 2007/054739, WO 2007/110667, WO 2007/116228, WO 2007/125364, WO 2008/097538, WO 2008/099210 and WO 2008/100412.

US 2006/0004013 and WO 2006/046575 disclose cinnamide derivatives which inhibit production of Aβ. The compounds are said to reduce the production of both Aβ(1-40) and Aβ(1-42). Related cinnamide derivatives are disclosed in US 2007/0117798, US 2007/0219181, WO 2007/135969 and WO 2007/135970.

Further compounds which are claimed to modulate Aβ levels are disclosed in WO 2004/110350.

The compounds of the present invention selectively attenuate production of Aβ(1-42).

According to the invention there is provided a compound of formula I:

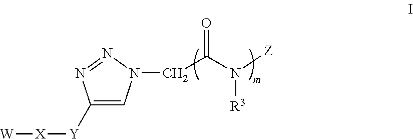

or a pharmaceutically acceptable salt or hydrate thereof; wherein:

W represents phenyl or 5- or 6-membered heteroaryl, any of which is optionally fused to a further 5- or 6-membered carbocyclic or heterocyclic ring, W optionally bearing up to 3 R¹ substituents;

each R¹ independently represents halogen, OH, amino, CF₃, C₁₋₄alkylamino, di(C₁₋₄alkyl)amino, C₂₋₆acylamino, N—C₁₋₄alkoxycarbamoyl, C₁₋₆alkoxy, C₁₋₆allylcarbonyl, or C₁₋₆alkyl which is optionally substituted with OH or C₁₋₄alkoxy;

X represents a bond, (CH₂)ₙO, (CH₂)ₙNH, CO or (CH₂)ₙNHCO where each n is 0 or 1;

Y represents a phenyl or 5- or 6-membered heteroaryl ring which optionally bears up to 3 R² substituents;

or the moiety W—X—Y may represent a fused-ring system consisting of 2 or 3 fused rings each of which is independently 5- or 6-membered and at least one of which is aromatic, said fused-ring system optionally bearing up to 3 R² substituents;

with the proviso that if X is a bond and W represents an imidazole, triazole or pyrazole ring which is linked to Y through N, then Y does not represent

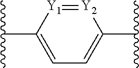

where Y1 and Y2 each independently represents N or CR²;

each R² independently represents halogen, CN, OH, C₁₋₆alkyl, or C₁₋₆alkoxy, said alkyl and alkoxy optionally having up to 3 fluorine substituents or a cyclopropyl substituent;

R³ represents H or polyfluoroC₁₋₆alkyl, or C₁₋₆alkyl which is optionally substituted with halogen, CN, OH, C₁₋₄alkoxy, phenyl or C₃₋₆cycloalkyl;

m is 0 or 1; and

Z is selected from:
(a) diphenylmethyl, C₃₋₁₀cycloalkyl or C₃₋₁₀cycloalkenyl, said C₃₋₁₀cycloalkyl or C₃₋₁₀cycloalkenyl optionally having up to 2 benzene rings fused thereto;
(b) phenyl which is optionally covalently linked to a further aromatic group containing up to 10 ring atoms, and
(c) phenyl which forms part of a fused ring system containing up to 2 additional rings, each of which independently comprises 5, 6 or 7 members and is independently carbocyclic or heterocyclic;

the group represented by Z optionally bearing up to 4 substituents independently selected from halogen, CN, NO₂, OH, oxo, C₁₋₄alkyl, C₂₋₄alkenyl, polyfluoroC₁₋₄alkyl, C₃₋₄cycloalkyl, C₃₋₆cycloalkylC₁₋₄alkyl, C₁₋₄alkoxy, C₁₋₄alkoxyC₁₋₄alkyl, polyfluoroC₁₋₄alkoxy, C₁₋₄alkylcarbonyl, polyfluoroC₁₋₄alkylcarbonyl, C₁₋₄alkoxycarbonyl, C₁₋₄alkylsulfonyl, SO₂NR₂, CONR₂, NR₂ and R₂N—C₁₋₄alkyl where each R is independently H or C₁₋₄alkyl or the two R groups together with the nitrogen to which they are attached complete a ring selected from azetidine, pyrrolidine, piperidine, piperazine and morpholine.

Where a variable occurs more than once in formula I, the identity taken by said variable at any particular occurrence is independent of the identity taken at any other occurrence.

As used herein, the expression “C₁₋ₓalkyl” where x is an integer greater than 1 refers to straight-chained and branched alkyl groups wherein the number of constituent carbon atoms is in the range 1 to x. Particular alkyl groups are methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₁₋₆alkoxy” are to be construed in an analogous manner.

The expressions “polyfluoroalkyl” and “polyfluoroalkoxy” refer to alkyl and alkoxy groups respectively in which one or more of the hydrogen atoms is replaced by fluorine, and includes embodiments of such groups in which all the hydrogens are replaced by fluorine. Examples thus include CH₂F, CHF₂, CF₃, CF₂CF₃, CH₂CF₃ and OCF₃.

The expression “C₃₋ₓcycloalkyl” where x is an integer greater than 3 refers to saturated cyclic hydrocarbon groups containing from 3 to x ring carbons. Where the value of x so permits, polycyclic systems containing fused rings and/or bridged bicyclic structures are included. Examples include cyclopropyl, cyclobutyl, cyclopentyl, cyclohexyl, decahydronaphthyl, bicyclo[2.2.2]octyl and adamantyl. “C₃₋ₓcycloalkenyl” similarly refers to nonaromatic unsaturated cyclic hydrocarbon groups, such as cyclopentenyl and cyclohexenyl.

The term “heterocyclic” refers to ring systems in which at least one ring atom is selected from N, O and S, the remaining ring atoms being carbon. Unless indicated otherwise, the term includes both saturated and unsaturated systems, including aromatic systems. Heterocyclic groups may be bonded via a ring carbon or a ring nitrogen, unless otherwise indicated. “Heteroaryl” refers to a heterocyclic ring that is aromatic.

The term “halogen” as used herein includes fluorine, chlorine, bromine and iodine, of which fluorine and chlorine, and in particular fluorine, are preferred unless otherwise indicated.

For use in medicine, the compounds of formula I may be in the form of pharmaceutically acceptable salts. Other salts may, however, be useful in the preparation of the compounds of formula I or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulfonic acid, benzenesulfonic acid, fumaric acid, maleic acid, succinic acid, acetic acid, benzoic acid, oxalic acid, citric acid, tartaric acid, carbonic acid or phosphoric acid. Alternatively, a pharmaceutically acceptable salt may be formed by neutralisation of a carboxylic acid group with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

It is to be understood that all the stereoisomeric forms encompassed by formula I, both optical and geometrical, fall within the scope of the invention, singly or as mixtures in any proportion.

Where a structure in accordance with formula I is capable of existing in tautomeric keto and enol forms, both of said forms fall within the scope of the invention, singly or as mixtures in any proportion.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

In formula I, X is a linking group selected from a bond, (CH₂)ₙO, (CH₂)ₙNH, CO and (CH₂)ₙNHCO where each n is 0 or 1; or X together with W and Y forms a fused ring system as described hereinafter. For the avoidance of doubt, when X represents CH₂O, CH₂NH or CH₂NHCO, W is attached to the CH₂ group. In a particular embodiment X represents a bond, O or NH, and more particularly X is a bond.

W represents phenyl or 5- or 6-membered heteroaryl, any of which is optionally fused to a further 5- or 6-membered carbocyclic or heterocyclic ring, and optionally bears up to 3 R¹ substituents. When W represents a heteroaryl ring, said ring typically comprises up to 3 heteroatoms selected from N, O and S. When W comprises an additional fused ring, said fused ring typically contains 0, 1 or 2 heteroatoms selected from N, O and S. In one sub-embodiment, W represents phenyl or a fused derivative thereof such as naphthalene, tetrahydronaphthalene, quinoline or methylenedioxyphenyl. In an alternative sub-embodiment W represents 6-membered heteroaryl such as pyridine, pyridazine or pyrimidine, or a fused derivative thereof such as quinoline. In a further sub-embodiment W represents 5-membered heteroaryl such as pyrrole, furan, thiophene, pyrazole, imidazole, oxazole, isoxazole, thiazole, isothiazole, triazole, oxadiazole or thiadiazole, or (where such fusion is feasible) the benzo- or pyrido-fused analogues thereof. In all of the aforementioned sub-embodiments, W optionally bears up to 3 (preferably up to 2) R¹ substituents where each R¹ independently represents halogen (e.g. F or Cl), OH, amino, CF₃, C₁₋₄alkylamino (e.g. methylamino), di(C₁₋₄alkyl)amino (e.g. dimethylamino), C₂₋₆acylamino (e.g. acetylamino), N—C₁₋₄-alkoxycarbamoyl (e.g. N-methoxycarbamoyl), C₁₋₆alkoxy (e.g. methoxy or ethoxy), C₁₋₆alkylcarbonyl (e.g. acetyl), or C₁₋₆alkyl which is optionally substituted with OH or C₁₋₄alkoxy (e.g. methyl, ethyl, isopropyl or hydroxymethyl). If two or more substituents are present, preferably not more than one of them is other than halogen, methoxy or C₁₋₆alkyl.

Specific examples of groups represented by W include pyridyl (for example 4-pyridyl) and pyrazolyl, either of which is optionally substituted with C₁₋₄alkyl (for example methyl).

Y represents a phenyl or 5- or 6-membered heteroaryl ring which optionally bears up to 3 (preferably up to 2) R² substituents. Suitable 6-membered heteroaryl rings include pyridine and pyrimidine, and suitable 5-membered heteroaryl rings include thiophene, furan, thiazole and imidazole. In the case of phenyl and 6-membered heteroaryl rings, the attachment points typically are in the 1,3 or 1,4 configuration (in particular 1,4), and in the case of 5-membered heteroaryl rings, the attachment points preferably are in the 1,3 configuration. Each R² independently represents halogen, CN, OH, C₁₋₆alkyl or C₁₋₆alkoxy, said alkyl and alkoxy optionally having up to 3 fluorine substituents or a cyclopropyl substituent. Suitable identities for R² include F, Cl, CN, methyl, methoxy, 2,2,2-trifluoroethoxy and cyclopropylmethoxy, in particular F, Cl, methyl and methoxy. In an embodiment of the invention, Y is a phenyl ring bearing a methoxy substituent ortho to the attachment point of W—X.

In a particular embodiment, W represents 4-pyridyl which optionally bears up to 2 substituents selected from F, Cl, CF₃, C₁₋₄alkyl, and C₁₋₄alkoxy, X is a bond, and Y represents 1,4-phenylene which optionally bears up to 2 substituents selected from F, Cl, CF₃, C₁₋₄alkyl, and C₁₋₄alkoxy.

In an alternative embodiment the moiety W—X—Y represents a fused-ring system consisting of 2 or 3 fused rings each of which is independently 5- or 6-membered and at least one of which is aromatic, said fused-ring system optionally bearing up to 3 (preferably up to 2) R² substituents. Suitable fused ring systems include quinoline, benzopyrazole, 1H-pyrrolo[2,3-c]pyridine, 4H-imidazo[2,1-c]-1,4-benzoxazine and [1]benzofuro[3,2-c]pyridine.

When m is 1 in formula I, R³ represents H or polyfluoroC₁₋₆alkyl (e.g. CF₃ or 2,2,2,-trifluoroethyl), or C₁₋₆alkyl which is optionally substituted with halogen, CN, OH, C₁₋₄alkoxy, phenyl or C₃₋₆cycloalkyl, for example methyl, ethyl, n-propyl, isopropyl, n-butyl, isobutyl, sec-butyl, tert-butyl, benzyl, cyclopropylmethyl, cyanomethyl, hydroxyethyl or methoxyethyl. In a particular embodiment m is 1 and R³ is 2,2,2,-trifluoroethyl, and in an alternative embodiment m is 0.

In one embodiment, Z represents diphenylmethyl, C₃₋₁₀cycloalkyl or C₃₋₁₀cycloalkenyl, said C₃₋₁₀cycloalkyl or C₃₋₁₀cycloalkenyl optionally having up to 2 benzene rings fused thereto. In a specific example of this embodiment, Z represents 9-fluorenyl.

In an alternative embodiment Z represents phenyl which is optionally covalently linked to a further aromatic group containing up to 10 ring atoms. Said further aromatic group, when present, may be 5- or 6-membered monocyclic or 8-, 9- or 10-membered fused bicyclic, and may be carbocyclic or heterocyclic, for example phenyl, pyridyl, thienyl, furyl, oxazolyl, imidazolyl, thiazolyl, pyrazolyl and the benzo- or pyrido-fused analogs thereof, e.g. benzoxazol-2-yl. In a sub-embodiment, said aromatic group is absent and Z represents a phenyl ring which is optionally substituted as described below.

In a further alternative, Z represents phenyl forming part of a fused ring system containing up to 2 additional rings, each of which independently comprises 5, 6 or 7 members and is independently carbocyclic or heterocyclic. Said fused ring or rings may be saturated or unsaturated. Most suitably, said fused ring or rings are carbocyclic. Examples of fused ring systems represented by Z include naphthalene, tetrahydronaphthalene, quinoline, anthracene and phenanthrene.

In each of the three embodiments described above, Z optionally bears up to 4 substituents independently selected from halogen, CN, NO₂, OH, oxo, C₁₋₄alkyl, C₂₋₄alkenyl, polyfluoroC₁₋₄alkyl, C₃₋₄cycloalkyl, C₃₋₆cycloalkylC₁₋₄alkyl, C₁₋₄alkoxy, C₁₋₄alkoxyC₁₋₄alkyl, polyfluoroC₁₋₄alkoxy, C₁₋₄alkylcarbonyl, polyfluoroC₁₋₄alkylcarbonyl, C₁₋₄alkoxycarbonyl, C₁₋₄alkylthio, C₁₋₄alkylsulfonyl, SO₂NR₂, CONR₂, NR₂ and R₂N—C₁₋₄alkyl where each R is independently H or C₁₋₄alkyl or the two R groups together with the nitrogen to which they are attached complete a ring selected from azetidine, pyrrolidine, piperidine, piperazine and morpholine. Preferred substituents include halogen, C₁₋₄alkyl and polyfluoroC₁₋₄alkyl, C₃₋₄cycloalkyl, C₃₋₆cycloalkylC₁₋₄alkyl, C₁₋₄alkoxy, C₁₋₄alkoxyC₁₋₄alkyl, polyfluoroC₁₋₄alkoxy, C₁₋₄alkylcarbonyl, C₁₋₄alkoxycarbonyl, C₁₋₄alkylthio and C₁₋₄alkylsulfonyl, and when 2 or more substituents are present preferably not more than one substituent is other than halogen, C₁₋₄alkyl or polyfluoroC₁₋₄alkyl.

Specific examples of groups represented by Z include 3,5-bis(trifluoromethyl)phenyl, 1-naphthalenyl, 4-tert-butylphenyl, 3-chloro-5-fluorophenyl, 4-(benzoxazol-2-yl)phenyl, 1,1,4,4,-tetramethyl-1,2,3,4-tetrahydronaphthalen-6-yl, 9-fluorenyl and phenanthren-9-yl.

Triazoles of formula I may be prepared by reaction of an azide of formula (1) with an alkyne of formula (2):

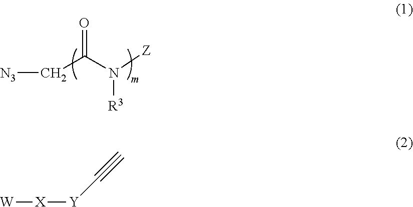

where Z, m, R³, W, X and Y have the same meanings as before. The reaction takes place at ambient temperature in aqueous ethanol or DMF in the presence of copper(II) sulphate and sodium ascorbate.

Azides (1) may be prepared from the corresponding halides by reaction with sodium azide in ethanol or DMF, or from the corresponding alcohols by reaction with diphenylphosphoryl azide in toluene in the presence of 1,8-diazabicyclo[5.4.0]undec-7-ene (DBU).

Compounds (2) in which X is a bond and Y is an aryl ring may be prepared by Suzuki coupling of boronic acid derivatives (3a) with aryl halides (4a):

W—B(OR)₂ (3a)

Hal-Y—≡ (4a)

or by analogous coupling of boronic acid derivatives (4b) with halides (3b):

W-Hal (3b)

(RO)₂B—Y—≡ (4b)

where each R independently represents H or C₁₋₆alkyl, or the two R groups complete a cyclic boronate ester such as pinacolate, Hal represents halogen (in particular iodine) and W and Y have the same meanings as before. The reaction takes place under standard Suzuki conditions, e.g. in a mixture of water, toluene and methanol with microwave heating in the presence of alkali metal carbonate and a triarylphosphine—Pd(0) catalyst.

Corresponding compounds (2) in which X is (CH₂)ₙNH are obtainable by coupling of amines (5):

W—(CH₂)ₙ—NH₂ (5)

with halides (4a), where W and n have the same meanings as before. The reaction takes place under Buchwald-Hartwig conditions, e.g. in a solvent such as DMF or t-amyl alcohol in the presence of strong base and a Pd-phosphine catalyst with microwave heating.

Similarly, compounds (2) in which X is (CH₂)ₙNHCO are obtainable by coupling of hydroxyl compounds (6):

W—(CH₂)ₙ—OH (6)

with halides (4a), where W and n have the same meanings as before. The reaction takes place with heating in the presence of cesium carbonate and cuprous chloride.

Corresponding compounds (2) in which X is (CH₂)ₙNHCO are obtainable by reaction of amines (5), halides (4a) and molybdenum hexacarbonyl, where W and n have the same meanings as before. The reaction takes place with heating in the presence of strong base (e.g. DBU), and a Pd-phosphine catalyst system.

In an alternative route to the compounds of formula I, alkynes (4a) or (4b) are reacted with azides (1) to form the triazole ring, followed by coupling with (3a), (3b), (5) or (6) as appropriate by the methods outlined above.

Where they are not commercially-available, the alkynes (4a) and (4b) may be obtained form the corresponding aldehydes by reaction with dimethyl (1-diazo-2-oxopropyl)phosphonate, e.g. in anhydrous methanol in the presence of anhydrous potassium carbonate. Alternatively, they may be obtained from the corresponding bromides by reaction with tributyl(ethynyl)tin or trimethylsilylacetylene in the presence of Pd(PPh)₄.

It will be readily apparent to those skilled in the art that individual compounds in accordance with formula I may be converted to further compounds of formula I using the normal techniques of organic synthesis such as oxidation, reduction, alkylation, condensation and coupling. For example, a compound of formula I in which R³ is H may be N-alkylated by conventional techniques to provide the corresponding compounds in which R³ is other than H. As an example there may be cited the treatment of a compound of formula I in which R⁹ is H with strong base (such as sodium hydride or caesium carbonate) followed by trifluoroethyl triflate to provide the N-trifluoroethyl derivative. Similar procedures may be carried out on intermediates such as the compounds (1).

Where they are not themselves commercially available, the starting materials for the synthetic schemes described above are available by straightforward chemical modifications of commercially available materials.

Certain compounds according to the invention may exist as optical isomers due to the presence of one or more chiral centres or because of the overall asymmetry of the molecule. Such compounds may be prepared in racemic form, or individual enantiomers may be prepared either by enantiospecific synthesis or by resolution. The novel compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallisation and regeneration of the free base. The novel compounds may also be resolved by formation of diastereomeric esters or amides, followed by chromatographic separation and removal of the chiral auxiliary. Alternatively, racemic intermediates in the preparation of compounds of formula I may be resolved by the aforementioned techniques, and the desired enantiomer used in subsequent steps.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} ed., 1999. The protecting groups may be removed at a convenient subsequent stage using methods known from the art.

The compounds of the invention have the useful property of modifying the action of γ-secretase on amyloid precursor protein so as to selectively reduce the formation of the 1-42 isoform of Aβ, and hence find use in the development of treatments for diseases mediated by Aβ(1-42), in particular diseases involving deposition of β-amyloid in the brain.

According to a further aspect of the invention there is provided the use of a compound according to formula I as defined above, or a pharmaceutically acceptable salt or hydrate thereof, for the manufacture of a medicament for treatment or prevention of a disease associated with the deposition of β-amyloid in the brain.

The disease associated with deposition of Aβ in the brain is typically Alzheimer's disease (AD), cerebral amyloid angiopathy, HCHWA-D, multi-infarct dementia, dementia pugilistica or Down syndrome, preferably AD.

In a further aspect, the invention provides the use of a compound of Formula I as defined above, or a pharmaceutically acceptable salt or hydrate thereof, in the manufacture of a medicament for treating, preventing or delaying the onset of dementia associated with Alzheimer's disease, cerebral amyloid angiopathy, HCHWA-D, multi-infarct dementia, dementia pugilistica or Down syndrome.

The invention also provides a method of treating or preventing a disease associated with deposition of Aβ in the brain comprising administering to a patient in need thereof a therapeutically effective amount of a compound of Formula I as defined above or a pharmaceutically acceptable salt or hydrate thereof.

In a further aspect, the invention provides a method of treating, preventing or delaying the onset of dementia associated with Alzheimer's disease, cerebral amyloid angiopathy, HCHWA-D, multi-infarct dementia, dementia pugilistica or Down syndrome comprising administering to a patient in need thereof a therapeutically effective amount of a compound of Formula I as defined above or a pharmaceutically acceptable salt or hydrate thereof.

The compounds of Formula I modulate the action of γ-secretase so as to selectively attenuate production of the (1-42) isoform of Aβ without significantly lowering production of the shorter chain isoforms such as Aβ(1-40) This results in secretion of Aβ which has less tendency to self-aggregate and form insoluble deposits, is more easily cleared from the brain, and/or is less neurotoxic. Therefore, a further aspect of the invention provides a method for retarding, arresting or preventing the accumulation of Aβ in the brain comprising administering to a subject in need thereof a therapeutically effective amount of a compound of Formula I as defined above or a pharmaceutically acceptable salt thereof.

Because the compounds of formula I modulate the activity of γ-secretase, as opposed to suppressing said activity, it is believed that the therapeutic benefits described above will be obtained with a reduced risk of side effects, e.g. those that might arise from a disruption of other signalling pathways (e.g. Notch) which are controlled by γ-secretase.

In one embodiment of the invention, the compound of Formula I is administered to a patient suffering from AD, cerebral amyloid angiopathy, HCHWA-D, multi-infarct dementia, dementia pugilistica or Down syndrome, preferably AD.

In an alternative embodiment of the invention, the compound of Formula I is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the compound of Formula I is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the compound of Formula I is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42),

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with nouns adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

The compounds of Formula I are typically used in the form of pharmaceutical compositions comprising one or more compounds of Formula I and a pharmaceutically acceptable carrier. Accordingly, in a further aspect the invention provides a pharmaceutical composition comprising a compound of formula I as defined above, or a pharmaceutically acceptable salt or hydrate thereof, and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage foams of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

For treating or preventing Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of 1 to 4 times per day. In some cases, however, a dosage outside these limits may be used.

The compounds of Formula I optionally may be administered in combination with one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which inhibit the secretion of Aβ (including γ-secretase inhibitors, β-secretase inhibitors, and GSK-3α inhibitors), compounds which inhibit the aggregation of Aβ, and antibodies which selectively bind to Aβ. Such additional compounds also include growth hormone secretagogues, as disclosed in WO 2004/110443.

In this embodiment of the invention, the amyloid modifier may be a compound which inhibits the secretion of Aβ, for example an inhibitor of γ-secretase (such as those disclosed in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621, and also including GSK-3 inhibitors, particularly GSK-3α inhibitors, such as lithium, as disclosed in Phiel et al, Nature, 423 (2003), 435-9.

Alternatively, the amyloid modifier may be a compound which inhibits the aggregation of Aβ or otherwise attenuates is neurotoxicicity. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 (in particular 3-aminopropane-1-sulfonic acid, also known as tramiprosate or Alzhemed™); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191. Further examples include phytic acid derivatives as disclosed in U.S. Pat. No. 4,847,082 and inositol derivatives as taught in US 2004/0204387.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466.

As used herein, the expression “in combination with” requires that therapeutically effective amounts of both the compound of Formula I and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the compound of Formula I.

### EXPERIMENTAL

The ability of the compounds of Formula I to selectively inhibit production of Aβ(1-42) may be determined using the following assay:

### Cell-Based γ-Secretase Assay

Human SH-SY5Y neuroblastoma cells overexpressing the direct γ-secretase substrate SPA4CT were induced with sodium butyrate (10 mM) for 4 hours prior to plating. Cells were plated at 35,000 cells/well/100 μl in 96-well plates in phenol red-free MEM/10% FBS, 50 mM HEPES, 1% Glutamine and incubated for 2 hrs at 37° C., 5% CO₂.

Compounds for testing were diluted into Me₂SO to give a ten point dose-response curve. Typically 10 μl of these diluted compounds in Me₂SO were further diluted into 182 μl dilution buffer (phenol red-free MEM/10% FBS, 50 mM HEPES, 1% Glutamine) and 10 μl of each dilution was added to the cells in 96-well plates (yielding a final Me₂SO concentration of 0.5%). Appropriate vehicle and inhibitor controls were used to determine the window of the assay.

After incubation overnight at 37° C., 5% CO₂, 25 μl and 50 μl media were transferred into a standard Meso avidin-coated 96-well plate for detection of Aβ(40) and Aβ(42) peptides, respectively. 25 μl Meso Assay buffer (PBS, 2% BSA, 0.2% Tween-20) was added to the Aβ(40) wells followed by the addition of 25 μl of the respective antibody premixes to the wells:

Aβ(40) premix: 1 μg/ml ruthenylated G2-10 antibody, 4 μg/ml; and biotinylated 4G8 antibody diluted in Origen buffer

Aβ(42) premix: 1 μg/ml ruthenylated G2-11 antibody, 4 μg/ml; and biotinylated 4G8 antibody diluted in Origen buffer

(Biotinylated 4G8 antibody supplied by Signet Pathology Ltd; G2-10 and G2-11 antibodies supplied by Chemicon)

After overnight incubation of the assay plates on a shaker at 4° C., the Meso Scale Sector 6000 Imager was calibrated according to the manufacturer's instructions. After washing the plates 3 times with 150 μl of PBS per well, 150 μl Meso Scale Discovery read buffer was added to each well and the plates were read on the Sector 6000 Imager according to the manufacturer's instructions.

Cell viability was measured in the corresponding cells after removal of the media for the Aβ assays by a colorimetric cell proliferation assay (CellTiter 96™ AQ assay, Promega) utilizing the bioreduction of MTS (Owen's reagent) to formazan according to the manufacturer's instructions. Briefly, 5 μl of 10×MTS/PES was added to the remaining 50 μl of media before returning to the incubator. The optical density was read at 495 nm after ˜4 hours.

LD₅₀ and IC₅₀ values for inhibition of Aβ(40) and Aβ(42) were calculated by nonlinear regression fit analysis using the appropriate software (eg. Excel fit). The total signal and the background were defined by the corresponding Me₂SO and inhibitor controls.

The compounds listed in the following examples all gave IC₅₀ values for Aβ(1-42) inhibition of less than 5 μM, in most cases less than 1.0 μM, and in many cases less than 0.5 μM. Furthermore, said values were at least 2-fold lower than the corresponding IC₅₀ values for Aβ(1-40) inhibition, typically at least 5-fold lower. The following table records IC₅₀ values for Aβ(1-42) inhibition for representative examples:

| Example No. | IC₅₀ Aβ(1-42) (nM) |
|---|---|
| 1 | 203 |
| 3 | 335 |
| 4 | 598 |
| 5 | 392 |
| 6 | 172 |

### Assay for In Vivo Efficacy

APP-YAC transgenic mice (20-30 g; 2-6 months old) and Sprague Dawley rats (200-250 g; 8-10 weeks old) are kept on 12-hr light/dark cycle with unrestricted access to food and water. Mice and rats are fasted overnight and are then dosed orally at 10 ml/kg with test compound formulated in either Imwitor:Tween-80 (50:50) or 10% Tween-80, respectively. For compound screening studies, test compounds are administered at a single dose (20 or 100 mg/kg) and blood taken serially at 1 and 4 hrs via tail bleed from mice and terminally at 7 hrs for mice and rats via cardiac puncture. In dose response studies, compounds are given at 0.1, 3, 10, 30, and 100 mg/kg and blood taken terminally at 7 hrs from mice and rats via cardiac puncture. Following euthanasia by CO₂, forebrain tissue is harvested from animals and stored at −80 degrees. For PD analysis of brain Aβ levels, soluble Aβ is extracted from hemi-forebrains by homogenization in 10 volumes of 0.2% DEA in 50 mM NaCl followed by ultracentrifugation. Levels of Aβ 42/40 are analyzed using Mesa Scale technology (electrochemiluminesence) with biotinylated 4G8 capture antibody and ruthenium labeled 12F4 or G210 detection antibodies for Aβ 42 and Aβ 40, respectively. For PK analysis, blood and brain samples are processed using a protein precipitation procedure with the remaining filtrate being analyzed via LC/MS/MS to determine drug exposure levels, brain penetration, and ED50/EC50, where appropriate.

Reductions in Aβ42 levels (relative to vehicle-treated controls) for representative compounds of the invention are in the range 50-90% whereas corresponding reductions in Aβ40 levels for the same compounds are 20-50%.

### Preparation of Starting Materials and Intermediates

General Procedure 1 (GP 1)—Preparation of Alkyl Azides from Alkyl Halides

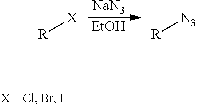

Alkyl halide (1.0 eq) and sodium azide (1.2-3 eq; caution, very toxic) were placed in a round bottom flask and ethanol or DMF (DMA, DMPU for unactivated and hindered substrates) were added to give an approximately 0.2 M solution. The mixture was stirred for 16 h at room temperature or up to 80° C. for less reactive electrophiles. The reaction mixture was then alternatively filtered through a 0.45 μM filter and used directly or more commonly worked-up according to the following procedure: water was added and the mixture extracted with methylene chloride twice. The combined organic layers were evaporated under reduced pressure without heating (caution: organic azides are explosive). The crude azide was used directly without further purification in the subsequent step according to GP3.

General Procedure 2 (GP2)—Preparation of Alkyl Azides from Alcohols

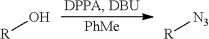

A 0.2 M suspension of alcohol (1.0 eq) and diphenylphosphoryl azide (1.3 eq) in toluene was treated with 1,8-Diazabicyclo[5.4.0]undec-7-ene (1.5 eq) at 0° C. slowly under N₂ atmosphere and the mixture slowly warmed to 23° C. The mixture was then stirred at this temperature for 16 hours. Once LCMS indicated complete consumption of the starting alcohol, the mixture was diluted with ethyl acetate and washed with water. The aqueous layer was extracted three times with ethyl acetate, and the organic layers were dried with sodium sulfate, filtered, and the solvent was removed under reduced pressure. The crude azide was used directly without further purification in the subsequent step according to GP3.

### General Procedure 3 (GP 3)—Preparation of Triazoles

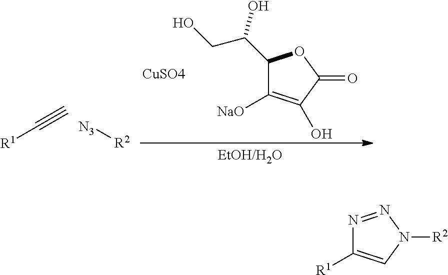

Alkyne, azide, water and ethanol (or DMF) were placed in a round bottom flask and aqueous copper sulfate solution (1M, 10-100 mol %) and aqueous sodium ascorbate solution (1M, 20-100 mol %) was added. The reaction mixture was stirred at room temperature until complete (more copper sulfate solution and sodium ascorbate solution were added as needed), then the solvents were removed under reduced pressure. The residue was purified by reversed phase chromatography (C18, acetonitrile/water with 0.1% TFA).

### Synthesis of 4-(4-Ethynyl-2-methoxy-phenyl)-2-methyl-pyridine

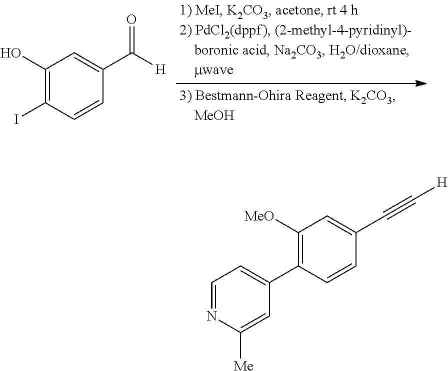

4-Iodo-3-methoxy-benzaldehyde: 3-Hydroxy-4-iodo-benzaldehyde (2 g, 8.06 mmol) and Potassium Carbonate (1.672 g, 12.10 mmol) were suspended in acetone (19.15 ml) and dimethylformamide (1.008 ml) at 23° C. and stirred for 10 minutes before iodomethane (0.756 ml, 12.10 mmol) was added via syringe. The resulting suspension was stirred at 25° C. for 4 hours. The reaction was concentrated under reduced pressure and washed with 250 ml water. The tan precipitate was collected via filtration, rinsed with water and dissolved in 400 ml ethyl acetate. This solution was washed with twice with 100 ml of brine, dried over sodium sulfate and filtered. Evaporation of the solvent gave 4-Iodo-3-methoxy-benzaldehyde (1.956 g, 7.46 mmol, 93% yield) as a tan solid.

¹H NMR (500 MHz, cdcl3) δ 9.93 (s, 1H), 7.96 (d, J=7.8, 1H), 7.27 (d, J=1.6, 1H), 7.16 (dd, J=7.8, 1.7, 1H), 3.94 (s, 3H).

MS (EI) [M+H]⁺ calc'd 263.0. Found 262.9.

3-Methoxy-4-(2-methyl-pyridin-4-yl)-benzaldehyde: 4-Iodo-3-methoxy-benzaldehyde (1.00 g, 3.82 mmol), (2-methyl-4-pyridinyl)-boronic acid (1.045 g, 7.63 mmol), PdCl2 (dppf) (0.558 g, 0.763 mmol) and sodium carbonate (0.809 g, 7.63 mmol) were dissolved in Dioxane (32 ml) and Water (8.00 ml), placed in a sealed tube and heated in a microwave reactor at 100° C. for 10 min. The reaction mixture was then filtered through a celite plug, which was rinsed with 50 mL methanol. The solution was concentrated and re-suspended in dichloromethane, washed with Brine and dried with magnesium sulfate. The residue was concentrated and purified by preparative HPLC Normal phase on silica gel, eluting with ethyl acetate/hexane (20%-100%), to give 3-Methoxy-4-(2-methyl-pyridin-4-yl)-benzaldehyde (510 mg, 2.244 mmol, 58.8% yield) as a dark solid.

¹H NMR (500 MHz, cdcl3) δ 10.03 (s, 1H), 8.55 (d, J=5.1, 111), 7.55 (dd, J=7.6, 1.1, 1H), 7.51 (s, 1H), 7.48 (d, J=7.6, 1H), 7.31 (s, 1H), 7.28 (d, J=5.1, 1H), 3.91 (s, 3H), 2.62 (s, 3H).

MS (EI) [M+H]⁺ calc'd 228.3. Found 228.1.

4-(4-Ethynyl-2-methoxy-phenyl)-2-methyl-pyridine: 3-Methoxy-4-(2-methyl-pyridin-4-yl)-benzaldehyde (510 mg, 2.244 mmol) and potassium carbonate (620 mg, 4.49 mmol) were placed in a nitrogen-flushed flask and methanol (14 mL) was added. Diethyl 1-diazo-2-oxopropylphosphonate (0.428 mL, 2.469 mmol) was added drop-wise to the heterogeneous solution, which was stirred at 23° C. for 16 hours. Reaction progress was monitored via LCMS. The yellow suspension was concentrated under reduced pressure, diluted with water and extracted with dichloromethane (10 mL) three times. The combined organic extracts were dried over magnesium sulfate, filtered and concentrated to afford 4-(4-Ethynyl-2-methoxy-phenyl)-2-methyl-pyridine (437 mg, 1.957 mmol, 87% yield) as a yellow oil. Further purification was not necessary.

¹H NMR (500 MHz, cdcl3) δ 8.51 (d, J=5.2, 1H), 7.29 (s, 1H), 7.25 (dd, J=5.4, 1.5, 1H), 7.19 (d, 1.4, 1H), 7.18 (d, J=1.4, 1H), 7.10 (d, J=1.2, 1H), 3.83 (s, 3H), 3.15 (s, 1H), 2.60 (s, 3H).

MS (EI) [M+H]⁺ calc'd 224.3. Found 224.1.

Other Alkynes were prepared by similar procedures

### Example 1

### 4-{4-[1-(3,5-Bis-trifluoromethyl-benzyl)-1H-1,2,3-triazol-4-yl]-2-methoxy-phenyl}-2-methyl-pyridine trifluoromethansulfonate salt

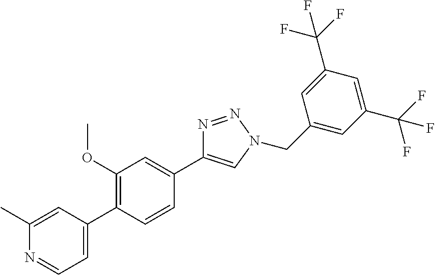

Following GP3, the title compound was prepared using 4-(4-ethynyl-2-methoxy-phenyl)-2-methyl-pyridine and 1-azidomethyl-3,5-bis-trifluoromethyl-benzene.

¹H NMR (500 MHZ, CDCl₃) δ 8.72 (s, 1H), 7.96 (s, 1H), 7.91 (s, 1H), 7.87 (d, J=5.1, 2H), 7.81 (m, 3H), 7.73 (s, 1H), 7.44 (s, 2H), 5.76 (s, 2H), 3.98 (s, 3H), 2.86 (s, 3H).

MS calculated 493.1 (MH⁺). Found 493.1 (MH⁺).

### Examples 2-13

The following were prepared by the same method, using the appropriate alkyne and azide:

| Example | Structure | MS (MH⁺) |
|---|---|---|
| 2 | 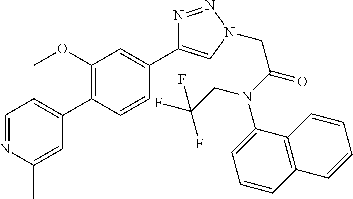 | Calc'd 532.2, found |
| 3 | 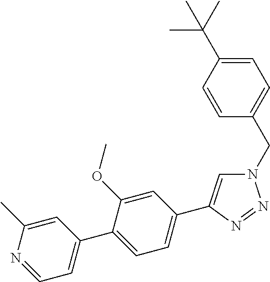 | Calc'd 413.2, found 413.2 |
| 4 | 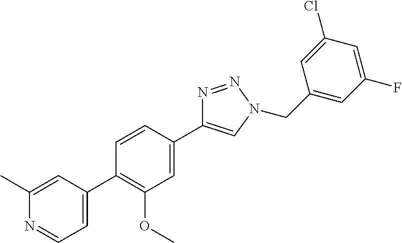 | Calc'd 409.1, found 409.1 |
| 5 | 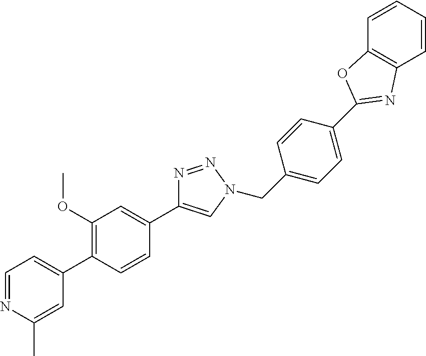 | Calc'd 474.2, found 474.1 |
| 6 | 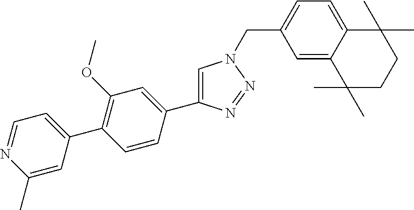 | Calc'd 467.3, found 467.2 |
| 7 | 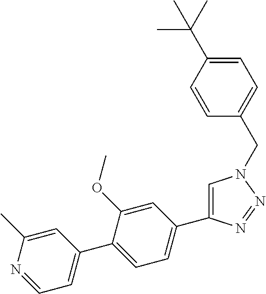 | Calc'd 413.2, found 413.2 |
| 8 | 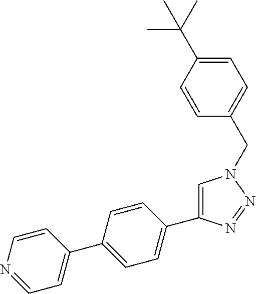 | Calc'd 369.2, found 369.2 |
| 9 | 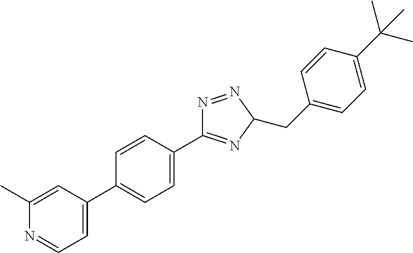 | Calc'd 383.2, found 384.2 |
| 10 | 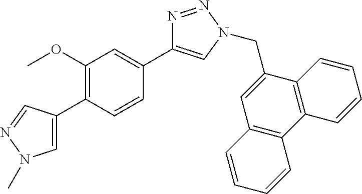 | Calc'd 446.5, found 446.1 |
| 11 | 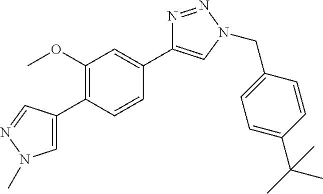 | Calc'd 402.5, found 402.1 |
| 12 | 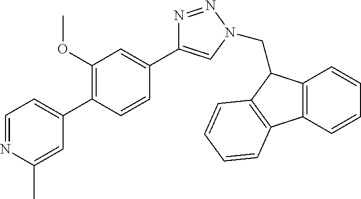 | Calc'd 445.5, found |
| 13 | 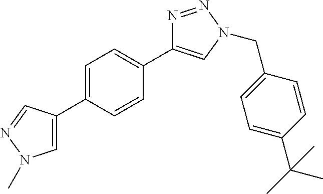 | Calc'd 372.2, found 372.2 |

1. A compound of formula I: 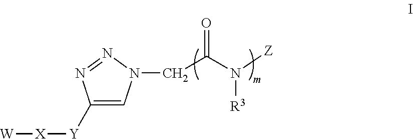
or a pharmaceutically acceptable salt or hydrate thereof; wherein:
W represents phenyl or 5- or 6-membered heteroaryl, any of which is optionally fused to a further 5- or 6-membered carbocyclic or heterocyclic ring, W optionally bearing up to 3 R¹ substituents;
each R¹ independently represents halogen, OH, amino, CF₃, C₁₋₄alkylamino, di(C₁₋₄alkyl)amino, C₂₋₆acylamino, N—C₁₋₄alkoxycarbamoyl, C₁₋₆alkoxy, C₁₋₆alkylcarbonyl, or C₁₋₆alkyl which is optionally substituted with OH or C₁₋₄alkoxy;
X represents a bond, (CH₂)ₙO, (CH₂)ₙNH, CO or (CH₂)ₙNHCO where each n is 0 or 1;
Y represents a phenyl or 5- or 6-membered heteroaryl ring which optionally bears up to 3 R² substituents;
or the moiety W—X—Y may represent a fused-ring system consisting of 2 or 3 fused rings each of which is independently 5- or 6-membered and at least one of which is aromatic, said fused-ring system optionally bearing up to 3 R² substituents;
with the proviso that if X is a bond and W represents an imidazole, triazole or pyrazole ring which is linked to Y through N, then Y does not represent 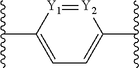
where Y1 and Y2 each independently represents N or CR²;
each R² independently represents halogen, CN, OH, C₁₋₆alkyl, or C₁₋₆alkoxy, said alkyl and alkoxy optionally having up to 3 fluorine substituents or a cyclopropyl substituent;
R³ represents H or polyfluoroC₁₋₆alkyl, or C₁₋₆alkyl which is optionally substituted with halogen, CN, OH, C₁₋₄alkoxy, phenyl or C₃₋₆cycloalkyl;
m is 0 or 1; and
Z is selected from:
(a) diphenylmethyl, C₃₋₁₀cycloalkyl or C₃₋₁₀cycloalkenyl, said C₃₋₁₀cycloalkyl or C₃₋₁₀cycloalkenyl optionally having up to 2 benzene rings fused thereto; and
(b) phenyl which is optionally covalently linked to a further aromatic group containing up to 10 ring atoms, and
(c) phenyl which forms part of a fused ring system containing up to 2 additional rings, each of which independently comprises 5, 6 or 7 members and is independently carbocyclic or heterocyclic;
the group represented by Z optionally bearing up to 4 substituents independently selected from halogen, CN, NO₂, OH, oxo, C₁₋₄alkyl, C₂₋₄alkenyl, polyfluoroC₁₋₄alkyl, C₃₋₄cycloalkyl, C₃₋₆cycloalkylC₁₋₄alkyl, C₁₋₄alkoxy, C₁₋₄alkoxyC₁₋₄alkyl, polyfluoroC₁₋₄alkoxy, C₁₋₄alkylcarbonyl, polyfluoroC₁₋₄alkylcarbonyl, C₁₋₄alkoxycarbonyl, C₁₋₄alkylthio, C₁₋₄alkylsulfonyl, SO₂NR₂, CONR₂, NR₂ and R₂N—C₁₋₄alkyl where each R is independently H or C₁₋₄alkyl or the two R groups together with the nitrogen to which they are attached complete a ring selected from azetidine, pyrrolidine, piperidine, piperazine and morpholine.

2. A compound according to claim 1 wherein Z represents 9-fluorenyl or phenyl, said phenyl optionally being fused to up to 2 additional 5-, 6- or 7-membered hydrocarbon rings, or optionally being covalently linked to a benzoxazole group.

3. A compound according to claim 1 wherein Z optionally bears up to 4 substituents independently selected from halogen, CN, C₁₋₄alkyl, CF₃, C₃₋₄cycloalkyl, C₁₋₄alkoxy, C₁₋₄alkoxyC₁₋₄alkyl, OCF₃, C₁₋₄alkylcarbonyl, C₁₋₄alkoxycarbonyl, C₁₋₄alkylthio, and C₁₋₄alkylsulfonyl.

4. A compound according to claim 1 wherein Y is a ring selected from phenyl, pyridine, pyrimidine, thiophene, furan, thiazole and imidazole and optionally bears up to 2 R² substituents.

5. A compound according to claim 4 wherein each R² independently represents H, F or C₁₋₄alkoxy.

6. A compound according to claim 1 wherein W represents phenyl, naphthalene, tetrahydronaphthalene, quinoline, methylenedioxyphenyl, pyridine, pyridazine, pyrimidine, or a 5-membered heteroaryl selected from pyrrole, furan, thiophene, pyrazole, imidazole, oxazole, isoxazole, thiazole, isothiazole, triazole, oxadiazole or thiadiazole, and a benzo- or pyrido-fused analogue of said 5-membered heteroaryl, any of which optionally bears up to two R¹ substituents.

7. A compound according to claim 6 wherein W represents 4-pyridyl which optionally bears up to 2 substituents selected from F, Cl, CF₃, C₁₋₄alkyl, and C₁₋₄alkoxy, X is a bond, and Y represents 1,4-phenylene which optionally bears up to 2 substituents selected from F, Cl, CF₃, C₁₋₄alkyl, and C₁₋₄alkoxy.

8. A pharmaceutical composition comprising a compound according to claim 1 or a pharmaceutically acceptable salt thereof and a pharmaceutically acceptable carrier.

9. A method of treating or preventing a disease associated with deposition of Aβ in the brain comprising administering to a patient in need thereof a therapeutically effective amount of a compound according to claim 1 or a pharmaceutically acceptable salt thereof.

10. A method as defined in claim 9 wherein the disease is Alzheimer's disease.
